(19) United States
(12) Patent Application Publication
(10) Pub. No.: US 2004/0237146 A1
(43) Pub. Date: Nov. 25, 2004

(54) Floral transformation

(76) Inventor: Thomas Leustek, New Brunswick, NJ (US)

Correspondence Address: Choate, Hall & Stewart, Exchange Place, 53 State Street, Boston, MA, 02109, US

(21) Appl. No.: 10/443,724

(22) Filed: May 22, 2003

(51) Int. Cl. A01H 1/00; C12N 15/82

(52) U.S. Cl. 800/294

(57) ABSTRACT

The present invention provides a system for transforming plants by floral wounding. Plant flowers are transformed by wounding of the floral bud and delivery of a bacterial suspension, such as Agrobacterium tumefaciens, carrying vector DNA. In preferred embodiments, the vector DNA includes nucleic acid encoding a pharmaceutical agent. Preferably, the flowers are allowed to mature to fruit and the seeds of the fruit are harvested and grown to produce mature transformed plants. The floral wounding methods of the present invention avoid the costly and time intensive step of plant regeneration required by standard transformation procedures.

### BACKGROUND

Current technology allows for the generation of genetically engineered plants by transforming plant cells with unique fragments of DNA that integrate into the plant genome. The most successful methods of plant transformation to date are those that utilize a species of Agrobacterium to deliver the transforming DNA; other available techniques include, for example, microprojectile bombardment or electroporation. Each of these techniques requires that a plant be regenerated from the transformed cells. However, the regeneration step has been problematic in several respects. The procedure is laborious and inefficient for many plant species. Numerous species are completely recalcitrant to transformation because it is not possible to grow their cells in culture. In addition, transformation of cell cultures can produce unanticipated and unwanted side effects such as phenotypic changes resulting from the tissue culture procedure itself, known as somaclonal variation. Because of these difficulties, scientists have been working to identify new ways to transform and regenerate recombinant plants.

Researchers have recently found that one plant species, Arabidopsis thaliana, can be successfully transformed without performing cell culture (Clough et al., Plant J. 16:735, 1998). Specifically, these researchers found that transformation could be accomplished by incubating A. tumefaciens with the Arabidopsis flower buds. Later work showed that transformation could be accomplished by dipping flowering Arabidopsis plants into a culture of the A. tumefaciens (Desfeux et al., Plant Physiol. 1123:895, 2000; Qing et al., Molecular Breeding: New Strategies in Plant Improvement 1:67, 2000; Curtis and Nam, Transgenic Research 10:363, 2001).

When researchers investigated the mechanism that allowed flowering Arabidopsis plants to become transformed when dipped into Arabidopsis culture, they found that the process by which Arabidopsis flowers develop passes includes a discrete stage in which a pore is present in the ovary wall. Specifically, they found that the gynoecium exists as an open, vase-like structure up to approximately 3 days prior to anthesis then ultimately fuses to form closed locules (Ye et al., Plant J. 19:249, 1999). If the flower is dipped into an Agrobacterium culture precisely during this stage, the Agrobacterium can pass through the pore and gain access to the interior of the ovary. Once inside the ovary, the Agrobacterium proliferates and transforms individual ovules (Desfeux et al., Plant Physiology 123:895, 2000). The transformed ovules follow the typical pathway of seed formation within the ovary to produce transgenic seeds. Transformed plants are identified from the population of seeds generated by the “dipped” flowers. This method provides a frequency of transformation of approximately 0.5%-1% for Arabidopsis, and produces independent transformation events, even among seeds derived from the same pod.

Recently, a floral dip transformation method was reported for Chinese cabbage (Brassica campestris L. ssp. Chinensis (pakchoi)), a species that is very closely related to Brassica juncea (Liu et al., Acta Hortic. (1998) 467:187-192). However, the efficiency was only 0.01%.

There exists a continued need for the development of efficient plant transformation strategies. There is a particular need for the development of techniques and reagents for transforming flowering plants.

### SUMMARY OF THE INVENTION

The present invention provides a system for transforming plants by floral wounding. In general, a plant is provided that is at the flowering stage. Preferably, the flowers are closed floral buds that have not yet opened. According to the present invention, the flowers are wounded and the wounded flower is contacted with a bacterial suspension carrying a nucleic acid. Wounding of the flower can be accomplished by any means that will result in the bacterial suspension being delivered to the interior, preferably the gynoecium, of the flower. Particularly preferred methods of wounding are mechanical methods, such as piercing with a needle or micropipette. In these particular embodiments, the bacterial suspension may be delivered via the piercing device (e.g., using a syringe or similar device).

Once the bacterial suspension is delivered to the flower, the flower is allowed to mature into a fruit. Mature fruit is collected and the seeds harvested. Some of the seeds will contain the transformed DNA. The seeds are grown to mature plants and the plants that were transformed with the nucleic acid expression vector are identified (e.g., by herbicide resistance, polymerase chain reaction, sequencing etc.).

According to one preferred embodiment of the invention, floral wounding transformation is accomplished by piercing a flower bud completely through with a needle to produce a hole on the opposite side of the entry point; retracting the needle to a position where the needle is within the flower bud; delivering a bacterial suspension (e.g., of Agrobacterium tumefaciens) carrying a nucleic acid vector to the flower bud until the suspension emerges from the hole on the opposite side of the entry point; and withdrawing the needle from the flower bud.

The floral wounding method of the present invention is amenable to plants having small and large flowers in the size range of 100 nanometers to 20 or even 30 millimeters in length. The present invention provides a transformation system useful for transforming plants not previously susceptible to efficient (or any) transformation. For example, in certain embodiments, the floral wounding methods of the present invention are used to transform fruit trees, which have, until now, have been difficult to genetically engineer. Other preferred plants to be transformed by the inventive floral wounding method include edible plants.

### BRIEF DESCRIPTION OF THE DRAWING

FIG. 1 is a diagram depicting the parts of a flower, particularly showing the gynoecium.

FIG. 2 is an illustration showing the floral wounding method.

FIG. 3 is an illustration showing the morphology of siliques resulting from floral wounding.

### DEFINITIONS

Expression of a gene refers to production of a gene product (e.g., primary transcript, mRNA, pre-protein, protein, higher order complex) and can involve one or more of the processes of transcription, splicing, RNA processing, translation, protein traficking, post-translational modification, protein folding, and/or oligomerization.

Expression cassette or expression vector refers to a nucleic acid molecule that includes one or more control elements sufficient to direct expression of another sequence.

Flowering stage refers to the stage of a flowering plant at which flower buds are beginning to open. Some of the flower buds will be open or beginning to open and some of the flower buds will be closed. Some plants form inflorescences or clusters of flower buds at the flowering stage.

A food or food product is any liquid or solid material appropriate for ingestion by humans or other animals. In some embodiments of the invention, edible plants or plant portions are transformed, and the transformed plant tissue is incorporated into a food or food product. In certain preferred embodiments, such a food or food product comprises raw plants or plant tissue. Alternatively, a plant-based food or food product may be prepared by any known method, including, for example, concentration or condensation of solid plant matter to fonn, for example, a pellet; production of a paste; drying, or lyophilization; cutting, mashing, or grinding the plant to various extents; or extraction of the liquid part of the plant to produce a soup, a syrup, or a juice. A processing step can also include cooking, e.g., steaming, the plant.

A gene, as that term is used herein, refers to an expressible portion of a nucleic acid. Typically, genes will be associated with regulatory sequences such as promoter sequences, 5′ or 3′ untranslated sequences, and termination sequences. In addition, introns and exons may also be included. The gene and regulatory sequences may be derived from the same natural source, or may be heterologous to one another. In certain preferred embodiments of the invention, a gene includes coding sequence for a polypeptide or protein.

Heterologous, as applied to nucleic acid sequences herein, means of different origin. For example, if a host cell is transformed with a nucleic acid sequence that does not occur in the untransformed host cell in nature, then that nucleic acid sequence is said to be heterologous to the host cell. Furthermore, different elements (e.g., promoter, enhancer, coding sequence, terminator, etc) of a transforming nucleic acid may be heterologous to one another and/or to the transformed host. The term heterologous, as used herein, may also be applied to nucleic acids that are identical in sequence to a nucleic acid already present in a host cell, but that are now linked to different additional sequences and/or are present at a different copy number, etc.

Mature plants are plants that have reached the reproductive stage and have produced flowers, which will become fruit that produce seeds.

A pharmaceutically active agent, as that term is used herein, is one that aids or contributes to the condition of a recipient in a positive manner when administered in a therapeutically effective amount. For example, a pharmaceutically active agent may have curative or palliative properties against a disease, and/or may be administered to ameliorate relieve, alleviate, reverse or lessen the severity of a disease or disorder. Alternatively or additionally, a pharmaceutically active agent may have prophylactic properties and/or may be used to prevent the onset of a disease or to lessen the severity of such disease or pathological condition when it does emerge. Pharmaceutically active agents may include an entire protein or polypeptide or instead may include only pharmaceutically active fragments thereof. The term also encompasses pharmaceutically active analogs of the agent. Furthermore, the term may also be used to refer to a single entity (e.g., small molecule, protein, or plant), or to a collection or complex of entities, for example that act cooperatively or synergistically to provide a therapeutic benefit.

A promoter, as used herein, is a DNA sequence that initiates transcription of an associated DNA sequence. A promoter region may also include elements that act as regulators of gene expression such as activators, enhancers, and/or repressors.

Regulatory elements refer to sequences involved in conferring the expression of a nucleotide sequence. Regulatory elements include 5′ regulatory sequences such as promoters that can be linked to the nucleotide sequence of interest, 3′ sequences such as 3′ regulatory sequences or termination signals. Regulatory elements also typically encompass sequences required for proper splicing, translation, trafficking, and/or modification of expression products.

Isolated orpurified is used to describe a protein, polypeptide, small molecule, or nucleic acid, etc, that is separated from at least one component with which it is associated when produced or in nature. As used herein, a protein, polypeptide, small molecule, or nucleic acid, etc., is said to be “isolated” or “purified” when it is substantially free of cellular material when it is isolated from recombinant and non-recombinant cells, or free of chemical precursors or other chemicals when it is chemically synthesized. In several contexts and typically refers to the at least partial purification of a protein, polypeptide, small molecule, nucleic acid, etc. away from unrelated or contaminating components (for example, plant structural and metabolic proteins). For example, an “isolated” or “purified” polypeptide or protein is substantially free of cellular material or other contaminating proteins from the cell or tissue source from which the protein is derived, or substantially free from chemical precursors or other chemicals when chemically synthesized. In one embodiment, the language “substantially free” means preparation of a protein having less than about 30%, 20%, 10% and more preferably 5% (by dry weight), of contaminating protein, or of chemical precursors or contaminating chemicals. A protein, polypeptide, small molecule or nucleic acid, etc, however, can be joined to another protein, polypeptide, small molecule, or nucleic acid with which it is not normally associated in a cell and still be considered “isolated” or “purified.” Methods for isolating and purifying proteins, polypeptides, small molecules, and nucleic acids are well known in the art.

Transformation refers to introduction of a nucleic acid into a cell, particularly the stable integration of a DNA molecule into the genome of an organism of interest.

Wounding refers to creating a disruption or breakage in a flower, particularly a flower bud. The wounding may be accomplished by any method that will introduce a disruption or breakage in flower bud that would not normally exist without physical contact or physical manipulation of the flower bud. For example, the wounding can occur by any number of techniques, which may include puncturing, poking, piercing, abrading, cutting, slicing, drilling, ripping, tearing, and the like. Various tools to perform the wounding may be used to achieve the desired effect, for example, needles, micropipettes, scissors, scalpels, and other such sharp objects.

### DETAILED DESCRIPTION OF THE INVENTION

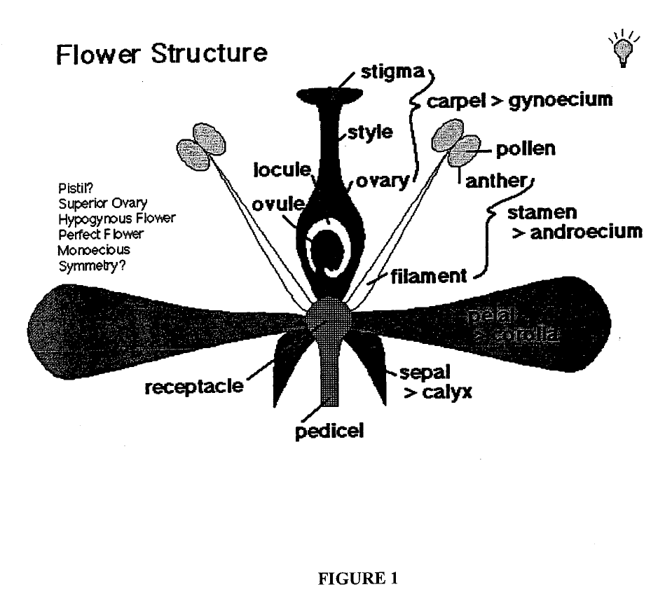

The present invention provides highly efficient methods for in planta transformation, which are based on the wounding of the flowers of a plant. Specifically, Agrobacterium carrying a nucleic acid expression vector is transferred into the gynoecium of a floral bud after the wounding is achieved (See FIG. 1). This new method results in increased levels of transformation. The new method is referred to herein as the “floral wounding method” of plant transformation.

Plants

According to the invention, plants having floral buds can be transfonned by the floral wounding, as described herein. Indeed, the floral wounding method of the invention may be applied to any species of plant used by man. Preferably, the species of plant is compatible with Agrobacterium. Such plants include crop plants, e.g., edible plants and plants that produce edible portions; ornamental plants, e.g., shrubs, flowers; industrial plants, e.g., waste removal plants, and the like.

Some preferred plants for use in accordance with the present invention are members of the plant family Brassicaceae. Members of this family include, but are not limited to cabbages, mustards, and radishes. The most preferred members of this family belong to the tribe Brassiceae. Members of this tribe include mustards of the family Brassica and related species, described in more detail below.

Certain preferred embodiments of the present invention utilize crop and/or crop-related members of the above-identified family and tribe. The term “crop member” refers specifically to species of the genus Brassica, which are commercially grown as sources for vegetables, oilseeds, forage, fodder, and condiments. Examples of crop members of the family Brassicaceae include, but are not limited to, digenomic tetraploids such as B. juncea (Indian mustard), B. carinata (Ethopian mustard), and B. napus (rapeseed); and monogenomic diploids such as B. oleracea (cole crops), B. nigra (black mustard), and B. campestris (turnip rape), Crambe abyssinica (oilseed crambe), Raphanus raphanistrum (radish), Rorippa sp. (watercress), Sinapis arvensis (wild mustard). Brassica juncea has great potential as a crop plant, a phytoremediation candidate, a delivery vehicle for micronutrients in the human diet, and as a factory for pharmaceutical proteins. Brassica oleraceae is used commercially for production of Canola vegetable oil. Other species and varieties of Brassica are popular commercial vegetables, including cabbage, broccoli, cauliflower, and brussel sprouts.

For the purposes of the invention, crop plants and/or crop related plants such as tomato, potato, and eggplant, which were previously only transformable by methods that involve the use of plant tissue cultures, can also be used in the present invention. Many plants can be regenerated from the culture of isolated vegetative tissue fragments can be transformed by the present invention. This includes but is not limited to all major species of plants that are edible for certain preferred embodiments, the plants are edible as sprouts. Some suitable plants include alfalfa, mung bean, radish, wheat, mustard, spinach, carrot, beet, onion, garlic, celery, rhubarb, a leafy plant such as cabbage or lettuce, watercress or cress, herbs such as parsley, mint, or clovers, cauliflower, broccoli, soybean, lentils, edible flowers such as the sunflower etc.

The floral wounding methods of the present invention broaden the species of plants amenable to genetic transformation considerably. For example, plants having large buds, such as fruit trees have been difficult or impossible to transform by standard methods. Such fruit trees include, for example, apple trees, peach trees, fig trees, orange trees, grapefruit trees, pear trees, plum trees, and the like. Any plants having large buds can now be transformed by the methods of the present invention.

Bacteria

The present invention relates to transformation of plant flowers with Agrobacterium. Agrobacterium is a representative genus of the gram-negative family Rhizobiaceae. Agrobacterium-based plasmid vectors allow the transformation of a wide range of plant species by capitalizing on the natural bacterial system for introducing DNA into the nuclear genome of plants. A. tumefaciens is a soil bacterium. It is pathogenic to a range of dicot plant species, causing the formation of crown galls or tumors at or close to infection sites. The proliferated tissue in the tumor provides the bacterium with unusual amino acids (opines), which are important to carbon and nitrogen source, at the expense of the host plant. Genes required to establish a tumor and bring about opine biosynthesis are transferred from Agrobacterium and hence this bacterium has been called Nature's genetic engineer.

Strains of Agrobacterium that are used with binary vector systems, which include two independent and complementing vectors. In this system, the T-DNA (transferred DNA) and vir genes reside on separated plasmids within the same Agrobacterium strain. The vir genes are located in a disarmed (without tumor forming genes) Ti plasmid and the T-DNA with the gene of interest is located in a small vector molecule. Of course, those skilled in the art will appreciate that not only binary vectors may be used in the present invention, but co-integrated vectors may also be used (Cormac et al., (1999) Mol. Gen. Genet. 261(2):226-235; Cormac et al. (1997) Mol. Biotechnol. 8(3): 199-213; Goodin et al., (2002) Plant J. 31(3):375-383; Goderis et al. (2002) Plant Mol. Biol. 50(1):17-27; Karimi eta l., (2002) Trends Plant Sci. 7(5):193-195; Kojima et al. (1999) DNA Res. 6(6):407-10; Hamilton et al., (1997) Gene 200(1-2):107-116; Hellens et al., Plant Mol. Biol. (2000) 42(6):819-832; Davey et al., Symp. Soc. Exp. Biol. (1986) 40:85-120; Davey et al., (1989) Plant Mol. Biol. 13(3):273-285; Potrykus I. (1990) Ciba Foundation Symp 154:198-208; Vain et al., (April 3, 2003) Theor. Appl. Genet. (epubl ahead of print); Wu et al., (2000) Genome 43(1):102-109; Xiang eta l., (1999) Plant Mol. Biol. 40(4):711-717).

Ti-vector-based plants are defined by their chromosomal background and resident Ti plasmid. Significant modifications to the virulence of Agrobacterium have expanded the range of plant species that are susceptible to T-DNA transformation by improving the frequency of T-DNA transfer. Although any of the numerous strains of A. tumefaciens capable of transferring genetic material to Brassica species can be used in combination with the other variations of the present invention, particularly improved transformation, recovery, and regeneration can be achieved by using A. tumefaciens strains GV3101, as well as other strains sharing common characteristics with these strains (for descriptions of various GV3101 strains see Table 1).

Ti-plasmids carried by A. tumefaciens may be engineered to include one or more sequences of interest to be introduced into a plant. Typically, engineered vectors will contain the sequences of interest inserted such that they are operatively linked to appropriate control sequences. For example, the bacterial genes responsible for opine expression, which are naturally carried on the Ti plasmid, can provide a convenient source of control elements to direct expression of inserted sequences. Relevant control sequences may include one or more of 1) promoter sequences; 2) 5′ untranslated sequences such as naturally occur in plants; 3) an initiation codon; 4) transcription tennination sequences; 5) translation termination sequences; 6) splicing control sequences, if relevant; 7) signal sequences that direct protein translocation and/or 8) sequences that affect post-translational processing of an encoded polypeptide.

Typical engineered vectors for introducing sequences into plants also include one or more of 1) a selectable or detectable marker (if the inserted sequences are not themselves readily detectable); 2) an origin of replication; and/or 3) various other sequences necessary and/or sufficient to direct Agrobacterium-mediated transformation of plants (e.g., virulence genes, which encode a set of proteins responsible for excision, transfer, and integration of T-DNA into a plant genome; transposon sequences or other sequences that allow homologous recombination with sequences in a plant genome, etc.). Such elements may be provided on a single vector or on multiple vectors; some elements may even be provided by the plant cell being transformed. Some exemplary useful sequences and vectors are described, for example, in Lawton et al., Plant Mol. Biol. 8:315, 1987; U.S. Pat. No. 5,888,789; Fraley et al., Proc. Natl. Acad. Sci. USA 80:4803, 1983; PCT/EP99/07414, each of which is incorporated herein by reference.

Transformation of Plants

According to the present invention, plants are first grown to the flowering stage. Preferably, floral wounding is carried out on young, closed flower buds that have not yet bloomed. Any open flowers on the plant are optionally removed from the plant, e.g., by pinching them off prior to floral wounding. A bacterium, e.g., Agrobacterium tumefaciens, carrying a nucleic acid expression vector, is then transformed into a plant by floral wounding. The flower may be wounded by any number of techniques. Preferably the wounding is accomplished by mechanical or physical wounding of the bud, which may include puncturing, poking, piercing, abrading, cutting, slicing, drilling, ripping, tearing, and the like. Various tools may be used to make the wound, for example, needles, micropipettes, scissors, scalpels, other sharp objects and the like.

In certain preferred embodiments, the bacteria carrying the nucleic acid expression vector are introduced into the floral bud simultaneously with the wounding. In other preferred embodiments, the bacteria are introduced into the floral bud after the wounding has been achieved (See FIG. 1). In one preferred embodiment, the ovary wall of flower bud is wounded using a needle. In certain embodiments, the needle is attached to a syringe, which is further filled with bacterial suspension of Agrobacteria lumefaciens (see, e.g., Qing et al., supra) carrying a nucleic acid expression vector encoding a protein of interest. The syringe is used to inject or inoculate the bacterial suspension into the floral bud through the needle once the wounding has occurred. Eventually, the Agrobacterium enter the gynoecium to transform the ovules.

The present invention is particularly amenable for use with floral buds that are relatively large, ranging in size from three to ten millimeters, or even larger flowers (within the size range of ten to 20 millimeters), which can be punctured with a typical syringe needle. The flower size range will vary depending on the delivery method. Most preferably, the Agrobacterium is delivered into the flower bud with the least possible damage to the flower bud. Larger flowers do not necessarily require larger needles. Small needles may be used with large flower buds to minimize damage to the bud. To provide but one example, a 260 gauge tuberculin syringe needle appropriate for larger flowers, would destroy flower buds less than one millimeter. A micropipette operated with a micromanipulator could also be used. Micropipettes and micromanipulators are well known in the art, particularly the biological arts. Those skilled in the art would appreciate that certain micropipettes and micromanipulators could easily be adapted for use with flower buds (see, e.g., Saitou eta l. (2000) Pflugers Arch 440(6):858-865; Costa et al. (1994) Biophys. J. 67(1):395-401; Kawase et al., (2002) Biol. Reprod. 66(2):381-385; Avila et al., (2001) Invest. Ophthalmol. Vis. Sci. 42(8):1841-1846; Attariwala et al., (1997) Invest. Opthamol. vis. Sci. 38(13):2742-2749;Glucksberg et al.(1993) Graefes Arch Clin Exp. Opthamol 231(7):405-407; Sherman et al., (1991) Methods in Enzym. 194:21-37)), could be used with flower buds in the size range of 100 nanometers to 1 millimeter or larger. Very large needles (for example, larger than 26 gauge) may be required for very large flowers, which are about 20 millimeters, to penetrate the tough wall of the ovary and reach the ovule within. For such purposes, any syringe needle that can pierce the flower bud without causing damage to the bud that results in spontaneous abortion of the bud can be used in the present invention. According to the present invention, the size of the needle is selected to cause the least amount of physical damage to the bud.

According to the present invention, the length of the needle may also be selected based on the length and/or width of the flower bud. For example, for particularly wide flower buds, a particularly long needle my be required to deliver the Agrobacterium into the flower bud. In certain preferred embodiments, the length of the needle may be selected to pierce all the way through the bud, in other preferred embodiments, the length of the needle may be selected to pierce partially through the flower bud.

In certain preferred embodiments, a micropipette may be used to deliver Agrobacterium tumefaciens cells carrying a binary TDNA expression vector to flowers of any size. In one preferred embodiment, a micropipette can be used to pierce a small or a large flower bud. The use of a micropipette may increase the transformation frequency of the floral wounding method since physical damage to the ovary would be minimized by precise delivery of the bacterial suspension by the micropipette to the gynoecium of the flower. This may further reduce the rate of spontaneous abortion of wounded floral buds. According to the present embodiment, plants of any species may be transfonned regardless of flower size.

In yet other preferred embodiments, smaller buds may first be wounded and then dipped into the bacterial suspension instead of physically placing the bacterial suspension into the bud by injection or other similar means. For example, a floral bud may be pierced or punctured, for example, by a micropipette or by a needle, and then submerged in a suspension of Agrobacterium tumefaciens carrying a binary T-DNA expression vector.

The flowers of Brassica juncea, a plant species of the Brassicaceae family, are relatively large, and are used to demonstrate the present invention (see Example 1). Agrobacterium tumefaciens carrying a binary T-DNA expression vector is introduced into young flower buds of B. juncea by wounding the ovary wall using a syringe needle filled with a bacterial suspension of Agrobacterium tumefaciens. In one exemplary embodiment, the needle is pierced completely through the flower bud at the thickest region of the bud and then retracted to a position where the needle orifice is within the flower bud. With the needle at this position, the bacterial suspension containing Agrobacterium tumefaciens is injected into the gynoecium of the flower. The bacterial suspension is delivered into the bud until the suspension emerges from the hole opposite the needle entry point. The needle is then carefully withdrawn.

Those skilled in the art will appreciate that any number of variations can be made to this transformation protocol. For example, in other preferred embodiments, instead of using a needle, a hole may be carefully drilled into the floral bud and then a needle inserted. Alternatively, a small incision may be made in the side of the floral bud.

Once the floral wounding method is complete, the plants are grown in potting soil to the flowering stage. Those of ordinary skill in the art would be well familiar with these methods. Briefly, the individual flowers are allowed to mature into fruits. Seeds are harvested from the fruits, planted and grown to mature plants, which potentially contain the transformed DNA. Those skilled in the art will appreciate that various selectable or detectable markers can be incorporated into the chosen expression vector to allow selection of transformed plants. One example of a selectable marker is a nucleic acid encoding an herbicide. Herbicides used for selection of genetically engineered plants are well known in the art, see, e.g., phosphenothricin, an inhibitor of glutamine synthase (Basta (Bayer Cropscience, Monheim am Rhein Germany), Finale (Farnum Companies Inc. Pheonix, Ariz.). Those skilled in the art will appreciate that there are many methods available to identify transformed plants, some other obvious examples include DNA sequencing and PCR (polymerase chain reaction), Southern blotting, RNA blotting, immunological methods for detection of a protein expressed from the DNA construct, e.g., precipitated protein that mediates phosphenothricin resistance, or other proteins such as reporter genes GUS, luciferase, green fluorescent protein (Harlow E., Lane, D. (1988) Antibodies: A Laboratory Manual. Cold Spring Harbor Laboratory Press, Cold Spring Harbor, N.Y. Ausubel et al., “Current Protocols in Molecular Biology”, Greene Publishing Associates, New York, V. 1&2 1996; Sambrook, J., E. Fritsch, and T. Maniatis, Molecular Cloning: A Laboratory Manual, Cold Spring Harbor Press, N.Y., both incorporated herein by reference)

Transforming plants by floral wounding and then growing the transformed plants to maturity is about 3 times faster than existing tissue culture transformation methods and requires less effort. This alone reduces the expense of generating the transgenic plants, compared to other standard methods, such as those requiring regeneration. In addition, although the total number of seeds produced by a wounded plant is lower than a non-wounded plant, of the seeds produced, 25% are typically transformed (see Table 2 in Example 1). This reduces the number of plants that have to be screened. Finally, although the plant aborts some of the wounded floral buds, the high frequency of transformation offsets this loss. For example, injection of Agrobacterium tumefaciens into as few as ten to twenty flowers produces many transformants. Using of herbicide resistance as a marker, the method is even more simple and facile.

Another factor that contributes to the low cost of the floral wounding method of the present invention is the equipment used. Not to limit the invention to these particular tools, materials such as needles and syringes, which are used in certain preferred embodiments of the invention, are inexpensive and easy to handle. Moreover, as noted above, the method of floral wounding further obviates the need to regenerate plants by the costly method of tissue culture.

Without limiting the invention, we propose that the introduction of Agrobacterium tumefaciens into flower buds results in bacteria gaining access to the interior of the ovary, where ovules are transformed with the expression vector. Although unclear, this may occur due to the damage to the ovary wall caused by the wounding. For example, where wounding is achieved by injection by a needle, the needle may damage the ovary wall when the flower bud is pierced. Alternatively, sperm cells within the developing pollen grains may be transformed, delivering the expression vector to the egg cell during pollination.

Nucleic acids

According to the present invention a nucleic acid is transformed into a plant by floral wounding. Any nucleic acid is suitable for the present invention. Those skilled in the art will appreciate that the nucleic acid may be a DNA or RNA. Preferably, the nucleic acid is detectable so that one can identify the plants that are transformed. Preferably, the nucleic acid includes an expressible unit included. In certain preferred embodiments, the expressible unit includes expressible sequences. In one preferred embodiment the expressible sequences encode a biologically active RNA or a biologically active protein. In one preferred embodiment, the protein is a pharmaceutically active agent, for example, a pharmaceutically active protein or RNA.

The protein of interest, according to the present invention may be any protein that one skilled in the art would want to express in a plant for. For example, the protein may be a plant protein or a mammalian protein. The present transformation method will be useful in expressing any of a multitude of traits or characteristics into crop, ornamental, or industrial plants. These might include disease resistance, stress resistance, increased yield, increased production efficiency, enhanced properties such as nutritional value, increased levels etc. Any heterologous nucleic acid, e.g., a heterologous nucleic acid encoding a protein of interest, may be contained within the nucleic acid transformed into the plant.

In certain preferred embodiments of the present invention, plants are transformed with sequences that encode one or more pharmaceutical proteins. Any pharmaceutical protein of interest may be expressed in plants in accordance with the present invention. Pharmaceutical proteins are proteins or polypeptides that either have pharmaceutical activity in animals, preferably in humans, or that produce, modify, or otherwise generate an agent that has pharmaceutical activity in animals, preferably humans. For instance, a protein that synthesizes or modifies a small molecule with pharmaceutical activity is a pharmaceutical protein in accordance with the present invention.

For example, particular pharmaceutical proteins of interest include, but are not limited to, hormones (insulin, thyroid hormone, catecholamines, gonadotrophines, trophic hormones, prolactin, oxytocin, dopamine, bovine somatotropin, leptins and the like), growth hormones (e.g., human growth hormone), growth factors (e.g., epidermal growth factor, nerve growth factor, insulin-like growth factor and the like), growth factor receptors, cytokines and immune system proteins (e.g., interleukins, colony stimulating factor (CSF), granulocyte colony stimulating factor (G-CSF), granulocyte-macrophage colony stimulating factor (GM-CSF), erythropoietin, tumor necrosis factor (TNF), interfersons, integrins, addressing, seletins, homing receptors, T cell receptors, immunoglobulins, soluble major histocompatibility complex antigens, immunologically active antigens such as bacterial, parasitic, or viral antigens or allergens), autoantigens, antibodies), enzymes (tissue plasminogen activator, streptokinase, cholesterol biosynthestic or degradative, steriodogenic enzymes, kinases, phosphodiesterases, methylases, de-methylases, dehydrogenases, cellulases, proteases, lipases, phospholipases, aromatases, cytochromes, adenylate or guanylaste cyclases, neuramidases and the like), receptors (steroid hormone receptors, peptide receptors), binding proteins (steroid binding proteins, growth hormone or growth factor binding proteins and the like), transcription and translation factors, oncoprotiens or proto-oncoprotiens (e.g., cell cycle proteins), muscle proteins (myosin or tropomyosin and the like), myeloproteins, neuroactive proteins, tumor growth suppressing proteins (angiostatin or endostatin, both which inhibit angiogenesis), anti-sepsis proteins (becteriocidal permeability-increasing protein), structural proteins (such as collagen, fibroin, fibrinogen, elastin, tubulin, actin, and myosin), blood proteins (thrombin, serum albumin, Factor VII, Factor VIII, insulin, Factor IX, Factor X, tissue plasminogen activator, Protein C, von Wilebrand factor, antithrombin III, glucocerebrosidase, erythropoietin granulocyte colony stimulating factor (GCSF) or modified Factor VIII, anticoagulants such as huridin) and the like.

The present invention also provides pharmaceutical proteins for veterinary use, such as vaccines and growth hormones, which may be produced by the transformed plants of the invention.

In certain embodiments of the present invention, pharmaceutical proteins are expressed in specific portions of plants, for example, roots, stems, flowers, leaves. In one embodiment, heterologous proteins are produced in edible portions of plants, for example by transforming edible plants and/or by placing relevant genes under control of tissue-specific regulatory sequences that direct expression in edible tissues. In such embodiments, the edible portions may be harvested and formulated into a pharmaceutical, for example, to be taken orally.

Pharmaceutical Compositions

Inventive transformed plants and/or the expression products of transformed sequences may be included in pharmaceutical compositions and/or administered to human or animal hosts in need thereof. Preferred hosts include vertebrates, preferably mammals, more preferably human. According to the present invention, the hosts include veterinary hosts such as bovines, ovines, canines, felines, etc. In one embodiment, the plant is edible at least as a sprout and can be administered orally to a host in a therapeutically effective amount. In other preferred embodiments, the pharmaceutically active agent is provided in a pharmaceutical preparation. Expression products (or products of expression products) of transformed sequences may be isolated an/or purified from plant tissues, and then may be formulated in to pharmaceutical compositions, as described herein.

The pharmaceutical preparations of the present invention can be administered in a wide variety of ways to the host, such as, for example, orally enterally, nasally, parenterally, intramuscularly or intravenously, rectally, vaginally, topically, ocularly, pulmonarily, or by contact application. In a preferred embodiment, a pharmaceutical agent expressed in a transgenic plant is administered to a host orally. In another preferred embodiment a pharmaceutically active agent expressed in a transgenic plant is extracted and/or purified, and used for the preparation of a pharmaceutical composition. Pharmaceutically active agents, for example, proteins that are enzymes (e.g., an enzyme that produces a small molecule), are isolated and purified in accordance with conventional conditions and techniques known in the art. These include methods such as extraction, precipitation, chromatography, affinity chromatography, electrophoresis, and the like.

The compositions of the present invention typically include an effective amount of a pharmaceutically active agent together with one or more organic or inorganic, liquid or solid, pharmaceutically suitable carrier materials. A pharmaceutically active agent produced according to the present invention is employed in dosage forms such as tablets, capsules, troches, dispersions, suspensions, solutions, capsules, creams, ointments, aerosols, powder packets, liquid solutions, solvents, diluents, surface active agents, isotonic agents, thickening or emulsifying agents, preservatives, solid bindings as long as the biological activity of the pharmaceutically active agent is not destroyed by such dosage form). Other examples of materials that can serve as pharmaceutically acceptable carriers include, but are not limited to sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening agents, flavoring agents, and perfuming agents, preservatives, and antioxidants can also be present in the composition, according to the judgment of the formulator (see also Remington's Pharmaceutical Sciences, Fifteenth Edition, E. W. martin (Mack Publishing Co., Easton Pa., 1975). For example, the pharmaceutically active agent may be provided as a pharmaceutical composition by means of conventional mixing granulating dragee-making, dissolving, lyophilizing, or similar processes.

In certain preferred embodiments it may be desirable to prolong the effect of a pharmaceutical preparation by slowing the absorption of the pharmaceutical agent that is subcutaneously or intramuscularly injected. This may be accomplished by the use of a liquid suspension of crystalline or amorphous material with poor water solubility. The rate of absorption of the agent then depends upon its rate of dissolution, which in turn, may depend upon size and form. Alternatively, delayed absorption of a parenterally administered agent is accomplished by dissolving or suspending the agent in an oil vehicle. Injectable depot forms are made by forming microencapsule matrices of the agent in biodegradable polymers such as polylactide-polyglycolide. Depending upon the ratio of the agent to the polymer and the nature of the particular polymer employed, the rate of release can be controlled. Examples of other biodegradable polymers include poly(orthoesters) and poly(anhydrides). Depot injectable formulations are also prepared by entrapping the agent in liposomes or microemulsions, which are compatible with body tissues.

Enterally administered preparations of pharmaceutically active agents may be introduced in solid, semi-solid, suspension or emulsion form and may be compounded with any pharmaceutically acceptable carriers, such as water, suspending agents, and emulsifying agents. The pharmaceutically active agents of the invention may also be administered by means of pumps or sustained-release forms, especially when administered as a preventive measure, so as to prevent the development of disease in a subject or to ameliorate or delay an already established disease.

The plants or pharmaceutically active agents produced according to the present invention are particularly well suited for oral administration as pharmaceutical compositions. The harvested plants may be processed in a variety of ways, e.g., air drying, freeze drying, extraction etc., depending on the properties of the desired product and the desired form of the final product. In preferred embodiments, such compositions as described above are ingested orally alone or ingested together with food or feed or a beverage. Compositions for oral administration include the plants themselves; extractions of the plants, and pharmaceutically active agents purified from the plants provided as dry powders, foodstuffs, aqueous or non-aqueous solvents, suspensions, or emulsions. Examples of non-aqueous solvents are propylene glycol, polyethylene glycol, vegetable oil, fish oil, and injectable organic esters. Aqueous carriers include water, water-alcohol solutions, emulsions or suspensions, including saline and buffered medial parenteral vehicles including sodium chloride solution, Ringer's dextrose solution, dextrose plus sodium chloride solution, Ringer's solution containing lactose or fixed oils. Examples of dry powders include any plant biomass that has been dried, for example, freeze-dried, air dried, or spray dried. The dried plants may be stored for further processing as bulk solids or further processed by grinding to a desired mesh sized powder. Alternatively, freeze-drying may be used for products that are sensitive to air-drying. Products may be freeze dried by placing them into a vacuum drier and dried frozen under a vacuum until the biomass contains less than about 5% moisture by weight. The dried material can be further processed as described herein.

Herbal preparations are well known in the art. Herbal preparations that may be used to administer the plants of the present invention include liquid and solid herbal preparations. Some examples of herbal preparations include tinctures, extracts (e.g., aqueous extracts, alcohol extracts), decoctions, dried preparations (e.g., air-dried, spray dried, frozen, or freeze-dried), powders (e.g., lyophilized powder), and liquid. Herbal preparations can be provided in any standard delivery vehicle, such as a capsule, tablet, suppository, liquid dosage, etc. Those skilled in the art will appreciate the various formulations and modalities of delivery of herbal preparations that may be applied to the present invention.

Those skilled in the art will appreciate that a particularly preferred method of obtaining the desired pharmaceutically active agent is by extraction. Fresh plants may be extracted to remove the desired products from the residual biomass, thereby increasing the concentration and purity of the product. Plants may also be extracted in a buffered solution. For example, the fresh harvested plants may be transferred into an amount of ice-cold water at a ratio of one to one by weight that has been buffered with, e.g., phosphate buffer. Protease inhibitors can also be added as required. The plants can be disrupted by vigorous blending or grinding while suspended in the buffer solution and the extracted biomass removed by filtration or centrifugation. The product carried in solution can be further purified by additional steps or converted to a dry powder by freeze-drying or precipitation. Pressing can also carry out extraction. Live plants can also be extracted by pressing in a press or by being crushed as they are passed through closely spaced rollers. The fluids expressed from the crushed plants are collected and processed according to methods well known in the art. Extraction by pressing allows the release of the products in a more concentrated form. However, the overall yield of the product may be lower than if the product were extracted in solution.

The plants, extractions, powders, dried preparations and purified products, etc., can also be in encapsulated form with or without one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active pharmaceutical agent may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

In other particularly preferred embodiments, a transgenic plant expressing a pharmaceutically active agent of the present invention or biomass of transgenic plants is administered orally as medicinal food. Such edible compositions are consumed by eating raw, if in a solid form, or by drinking, if in liquid form. In a preferred embodiment, the transgenic plant material is directly ingested without a prior processing step or after minimal culinary preparation. For example, the phannaceutically active agent, e.g., a protein, is expressed in a plant of which can be eaten directly. In an alternative embodiment, the plant biomass is processed and the material recovered after the processing step is ingested.

Processing methods preferably used in the present invention are methods commonly used in the food or feed industry. The final products of such methods still include a substantial amount of the expressed pharmaceutically active agent and are preferably conveniently eaten or drunk. The final product may also be mixed with other food or feed forms, such as salts, carriers, favor enhancers, antibiotics, and the like, and consumed in solid, semi-solid, suspension, emulsion, or liquid form. In another preferred embodiment, such methods include a conservation step, such as, e.g., pasteurization, cooking, or addition of conservation and preservation agents. Any plant is used and processed in the present invention to produce edible or drinkable plant matter. The amount of pharmaceutically active agent in an edible or drinkable plant preparation may be tested by methods standard in the art. For example, if the pharmaceutically active agent is a protein, gel electrophoresis, Elisa, or Western blot analysis, using an antibody specific for the protein. This determination is used to standardize the amount of protein ingested. For example, the amount of therapeutically active protein in a plant juice determined and regulated, for example, by mixing batches of product having different levels of protein so that the quantity of juice to be drunk to ingest a single dose can be standardized. The contained, regulatable environment of the present invention, however, should minimize the need to carry out such standardization procedures. This process can be performed on any phannaceutically active agent that may be included in or expressed by the transformed nucleic acid.

A pharmaceutically active agent produced in a plant and eaten by a host is absorbed by the digestive system. One advantage of the ingestion of a plants (e.g., plant parts, or sprouted seedling preparation) particularly intact plants or plant biomass that has been only minimally processed, is to provide encapsulation or sequestration of the agent in cells of the plant. Thus, the agent may receive at least some protection from digestion in the upper digestive tract before reaching the gut or intestine and a higher proportion of active would be available for uptake.

The pharmaceutical compositions of the present invention can be administered therapeutically or prophylactically. In certain preferred embodiments, the compositions may be used to treat or prevent a disease. For example, any individual who suffers from a disease or who is at risk of developing a disease may be treated. It will be appreciated that an individual can be considered at risk for developing a disease without having been diagnosed with any symptoms of the disease. For example, if the individual has a particular genetic marker identified as being associated with increased risk for developing a particular disease, that individual will be considered at risk for developing the disease. Similarly, if members of an individual's family have been diagnosed with a particular disease, e.g., cancer, the individual may be considered to be at risk for developing that disease.

Liquid dosage forms for oral administration include, but are not limited to, pharmaceutically acceptable emulsions, microemulsions, solutions, suspensions, syrups, and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Compositions for rectal or vaginal administration are preferably suppositories which can be prepared by mixing the compositions of this invention with suitable non-irritating excipients or carriers such as cocoa butter, polyethylene glycol or a suppository wax which are solid at ambient temperature but liquid at body temperature and therefore melt in the rectum or vaginal cavity and release the active agent.

Dosage forms for topical or transdermal administration of a pharmaceutical composition of this invention include ointments, pastes, creams, lotions, gels, powders, solutions, sprays, inhalants or patches. The active agent, or preparation thereof, is admixed under sterile conditions with a pharmaceutically acceptable carrier and any needed preservatives or buffers as may be required. Ophthalmic formulation, eardrops, and eye drops are also contemplated as being within the scope of this invention. Additionally, the present invention contemplates the use of transdermal patches, which have the added advantage of providing controlled delivery of a pharmaceutically active agent to the body. Such dosage forms can be made by suspending or dispensing the pharmaceutically active agent in the proper medium. Absorption enhancers can also be used to increase the flux of the pharmaceutically active agent across the skin. The rate can be controlled by either providing a rate controlling membrane or by dispersing the pharmaceutically active agent in a polymer matrix or gel.

The compositions are administered in such amounts and for such time as is necessary to achieve the desired result. As described above, in certain embodiments of the present invention a “therapeutically effective amount” of a pharmaceutical composition is that amount effective for treating, attenuating, or preventing a disease in a host. Thus, the “amount effective to treat, attenuate, or prevent disease”, as used herein, refers to a nontoxic but sufficient amount of the pharmaceutical composition to treat, attenuate, or prevent disease in any host. As but one example, the “therapeutically effective amount” can be an amount to treat, attenuate, or prevent diabetes.

The exact amount required will vary from subject to subject, depending on the species, age, and general condition of the subject, the stage of the disease, the particular pharmaceutical mixture, its mode of administration, and the like. The plants of the invention and/or preparations thereof are preferably formulated in dosage unit form for ease of administration and uniformity of dosage. The expression “dosage unit form,” as used herein, refers to a physically discrete unit of pharmaceutically active agent appropriate for the patient to be treated. It will be understood, however, that the total daily usage of the compositions of the present invention are preferably decided by an attending physician within the scope of sound medical judgment. The specific therapeutically effective dose level for any particular patient or organism will depend upon a variety of factors including the disorder being treated and the severity of the disorder; the activity of the specific compound employed; the specific composition employed; the age, body weight, general health, sex of the patient, diet of the patient, pharmacokinetical condition of the patient, the time of administration, route of administration, and rate of excretion of the specific compound employed; the duration of the treatment; drugs used in combination or coincidental with the specific compound employed; and like factors well known in the medical arts.

It will also be appreciated that the pharmaceutical compositions of the present invention can be employed in combination therapies, that is, the pharmaceutical compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another anti-cancer agent), or they may achieve different effects.

Equivalents

The representative examples that follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art. The following examples contain important additional information, exemplification, and guidance, which can be adapted to the practice of this invention in its various embodiments and the equivalents thereof.

### EXAMPLES

### Materials and Methods

Preparation of Agrobacterium tumefaciens for transformation

A. tumefaciens strain GV3101 carrying one of the plasmids listed in Table 1 was grown at 30° C. on Luria Broth (LB) agar for 2 days. The medium contained 50 mg L⁻¹ kanamycin and 20 mg L⁻¹ tetracycline. A single colony from each culture was grown at 30° C. for 1 day in liquid LB containing the same concentration of respective antibiotics and 100 mM acetophenone (Fluka) up to OD₆₀₀=0.6. The culture was centrifuge at 5000 rpm for 15 min. The pellet was resuspened in MS liquid 0.050% (v/v) Silwet L-77, pH5.7.

TABLE 1
Agrobacterium strains carrying plasmid constructs used in this study.

(1) GV3101 carrying pTT101 with the human growth hormone gene
under transcriptional control of the HSP18.2 promoter, the plasmid carries
a kanamycin selection marker
(2) GV3101 carrying pTT101 with the human insulin gene 1 under
transcriptional control of the HSP18.2 promoter, the plasmid carries a
kanamycin selection marker
(3) GV3101 carrying pTT01 with the human insulin gene 2 under
transcriptional control of the HSP18.2 promoter, the plasmid carries a
kanamycin selection marker
(4) GV3101 carrying pGreen with the phosphenothricin (BASTA ™)
selection marker (BAR gene)
(5) GV3101 carrying pGreen with the phosphenothricin (BASTA ™)
selection marker (BAR gene) and a DNA sequence of unknown origin

Agrobacterium-mediated transformation by injection into flowers.

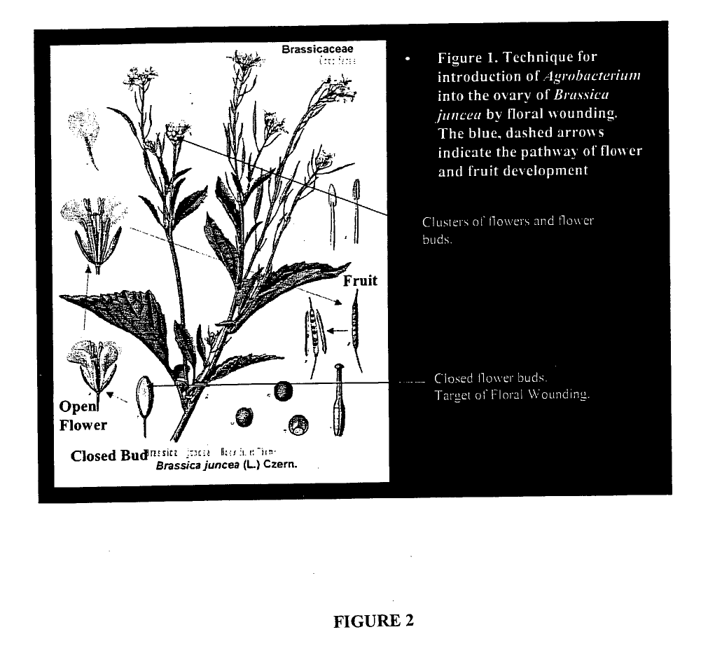

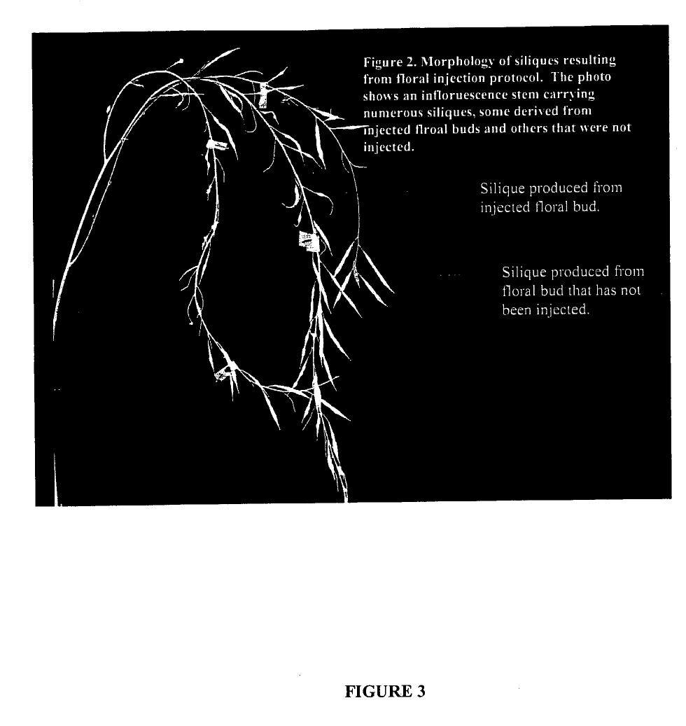

Brassica juncea seeds were germinated and the plants grown in potting mix, in a growth chamber maintained at 25°±1° C. under a 16/8 hours (light/dark) photoperiod for 15-20 days until the plants began flowering. The plants were watered regularly with a Jack's Classic Blossom Booster water-soluble nutrients (J.R. Peters, Inc., Allentown, Pa.). Plants were grown to the flowering stage as described above. Flower buds form in clusters at the ends of B. juncea inflorescences (FIG. 2). All opened flowers were removed from the cluster. The remaining closed flower buds range in length from 3 to 6 mm. Agrobacterium suspension was injected into the closed flower buds as follows. A syringe equipped with a 27-gauge tuberculin needle was filled with a suspension of freshly prepared Agrobacterium. The needle was used to pierce completely through the flower bud at the thickest region of the bud. Then the needle was retracted to a position where the needle orifice was within the flower bud. The Agrobacterium suspension (approximately 50 μL) was delivered until the suspension emerged from the hole opposite the needle entry point. The needle was then completely withdrawn, and the procedure repeated on another bud in the cluster. The plants were returned to the growth chamber to complete floral development. The procedure resulted in the spontaneous abortion of approximately 80% of pierced flower buds, due either to damage from the needle or to runaway growth of Agrobacterium within the bud. Of the buds that develop into flowers and produced fruits (siliques), evidence of damage from the procedure was observed as siliques that contain fewer numbers of seeds than normal (FIG. 3). Seeds were collected and transformation tested.

Selection of putative transgenic plants transformed with pTT101 vectors

The pTT 101 vector contains a kanamycin-resistance marker. To select for transformants seeds were germinated under axenic conditions. The seeds were surface sterilized in 100% commercial bleach solution (Clorox) for 1 min with gentle agitation. The seeds were rinsed three times with sterile ultrapure H₂O. They were then soaked in 95% (v/v) ethanol for 5 min with gentle agitation, and were finally rinsed three times with sterile ultrapure H2O.

Surface sterilized seeds were germinated in petri dishes on medium containing Murashigi and Skoog inorganic components (Sigma catalog number M5519) solidified with 8% (w/v) agar and supplemented with 100 mg/L kanamycin and 500 mg/L carbenicillin at 25°±1° C. under a 16/8 hour (light/dark) photoperiod. The seedlings are large and grow off the surface of the agar medium. Since most of the seedling body is not in contact with the medium, to prevent false transformants, putative transformants were exposed to a secondary and then tertiary round of antibiotic selection by determining whether the shoots would grow in the presence of antibiotics and then whether roots could be regenerated in the presence of antibiotics. Sixteen days after germination the root was removed from putative transformed plants by severing the hypocotyls. The shoot was transferred to MS medium with the hypocotyls implanted into the medium (Sigma, M5519) solidified with 8% (w/v) agar, and supplemented with 50 mg/L kanamycin, 500 mg/L carbenicillin and 20 g/L sucrose. The growing shoot tip was transferred twice, at 14-day intervals, to fresh medium.

Shoots that survived the second round of selection were tested for rooting ability in the presence of antibiotics. Growing shoots were transferred to rooting medium containing MS nutrients (Sigma, M5519) solidified with 8% (w/v) agar, and supplemented with 50 mg/L kanamycin, 500 mg/L carbenicillin and 20 g/L sucrose and 2 mg/L indolebutyric acid (a synthetic plant hormone that induces root formation).

Rooted plants were transferred to soil. The agar medium was washed from the roots with water. The plantlets were planted in moist potting mix and placed in a high humidity growth chamber at 25°±1° C. under a 16/8 hours (light/dark) photoperiod. The plants were slowly exposed to ambient humidity and transferred to a greenhouse.

Selection of putative transgenic plants transformed with pGREEN vector

The pGREEN vector contains the BAR gene for resistance to glufosinate, an herbicide sold under the commercial name BASTA™ or FINALE™. The advantage is that the herbicide can be sprayed directly onto seedlings. There is no need for in vitro selection of transformants. Seeds were germinated in potting mix and grown in a growth chamber at 25°±1° C. under a 16/8 hours (day/night) photoperiod for 15 days. Plants were sprayed with a 1000 fold dilution of FINALE™ (Farnman Inc, Poenix, Ariz., 5.78% glufosinate-ammonium CAS#77182-82-2) in water. Resistant plants were transferred to the greenhouse, grown to maturity, and seeds collected.

Identification of the transgene construct in putative transformants

A polymerase chain reaction was used to identify the transgenic construct in putative transformants. Specific primers were used to identify each of the constructs used.

### Results

The results from several transformation experiments are summarized in Table 2. The results indicate that the floral wounding method achieves a transformation efficiency of up to 25.5%. This is an extremely high efficiency compared to previously available methods.

TABLE 2
Transformation efficiency.

| | | | | No. tested for | | |
|---|---|---|---|---|---|---|
| | | No. of | No. of | DNA | No. positive | Percentage |
| Construct | Selection | Plants tested | resistant | construct | for construct | transformed |
| 1 | KAN | 300 | 50 | 12 | 4 | 8.0^{A} |
| 2 | KAN | 300 | 50 | 10 | 3 | 6.0^{A} |
| 3 | KAN | 300 | 50 | 25 | 5 | 10.0^{A} |
| 4 | BAR | 72 | 18 | 18 | 18 | 25.0^{B} |
| 5 | BAR | 72 | 18 | 18 | 18 | 25.5^{B} |

^{A}percentage of plants testing positive for the DNA construct compared with the number resistant to antibiotic,
^{B}percentage of plants testing positive for the DNA construct compared with the number tested for herbicide resistance. Because of the difficulty of selecting B. juncea for kanamycin resistance, the number of false positives is large. For this reason, transformation efficiency was estimated based on the number testing positive for the DNA construct.

### Other Embodiments

Those of ordinary skill in the art will appreciate that the foregoing represents certain preferred embodiments of the present invention and should not be construed to limit the spirit or scope of the invention as defined by the following claims:

We claim:

1. A method of transforming a plant, comprising:
providing a plant that is at the flowering stage and contains at least one flower;
wounding the flower of the plant; and
delivering a bacterial suspension carrying a nucleic acid expression vector to the flower.

2. The method of claim 1, further comprising allowing the flowers to mature into fruit.

3. The method of claim 2, further comprising collecting the seeds from the fruit.

4. The method of claim 3, further comprising growing the seeds to mature plants and identifying which plants are transformed with the nucleic acid expression vector.

5. The method of claim 1, wherein the flowers are young, closed buds.

6. The method of claim 1, wherein wounding comprises mechanical wounding.

7. The method of claim 6, wherein mechanical wounding comprises puncturing, poking, piercing, abrading, cutting, slicing, drilling, ripping, or tearing.

8. The method of claim 6, wherein mechanical wounding comprises piercing the flower with a needle.

9. The method of claim 6, wherein mechanical wounding comprises piercing the flower with a micropipette.

10. The method of claim 1, wherein the expression vector includes a nucleic acid encoding a pharmaceutical agent.

11. The method of claim 1, wherein the expression vector includes nucleic acid encoding a selectable or detectable marker.

12. The method of claim 11, wherein the selectable marker is an herbicide resistance gene.

13. The method of claim 1, wherein the bacterial suspension is delivered to the gynoecium of the flower.

14. The method of claim 1, wherein delivering comprises inoculating, spraying, or submerging.

15. The method of claim 1, wherein delivering comprises injecting.

16. The method of claim 1, wherein the bacterial suspension comprises Agrobacterium tumefaciens.

17. The method of claim 12, wherein identifying comprises growing the seeds in the presence of an herbicide to which the nucleic acid vector imparts resistance.

18. A method of transforming a flower comprising:
piercing a flower bud completely through with a needle to produce a hole on the opposite side of the entry point;
retracting the needle to a position where the needle is within the flower bud;
delivering a bacterial suspension carrying a nucleic acid vector to the flower bud until the suspension emerges from the hole on the opposite side of the entry point;
withdrawing the needle from the flower bud.

19. The method of claim 18, wherein the flower bud is a young, closed flower bud.

20. The method of claim 18, wherein the bacterial suspension is delivered to the gynoecium of the flower bud.

21. The method of claim 18, wherein the bacterial suspension contains Agrobacterium tumefaciens.

22. The method of claim 18, wherein the nucleic acid vector includes a nucleic acid encoding a pharmaceutical agent.

23. The method of claim 18, wherein the nucleic acid vector includes nucleic acid encoding a selectable or detectable marker.

24. The method of claim 23, wherein the selectable marker is an herbicide resistance gene.

25. The method of claim 18, wherein delivering comprises injecting the bacterial suspension using a syringe.

26. A method of transforming an edible plant, comprising:
providing an edible plant that is at the flowering stage;
wounding the flower of the plant; and
delivering a bacterial suspension carrying a nucleic acid vector to the flower.

27. The method of claim 26, further comprising allowing the flowers to mature into fruit.

28. The method of claim 27, further comprising collecting the seeds from the fruit.

29. The method of claim 28, further comprising growing the seeds to mature plants and identifying which plants are transformed with the nucleic acid vector.

30. The method of claim 29, further comprising growing seeds from mature transformed plants to sprouted seedlings.

31. The method of claim 26, wherein the flowers are young, closed buds.

32. The method of claim 26, wherein wounding comprises mechanical wounding.

33. The method of claim 32, wherein mechanical wounding comprises puncturing, poking, piercing, abrading, cutting, slicing, drilling, ripping, or tearing.

34. The method of claim 32, wherein mechanical wounding comprises piercing the flower with a needle.

35. The method of claim 32, wherein mechanical wounding comprises piercing the flower with a micropipette.

36. The method of claim 26, wherein the nucleic acid vector includes a nucleic acid encoding a pharmaceutical agent.

37. The method of claim 26, wherein the nucleic acid vector includes nucleic acid encoding a selectable or detectable marker.

38. The method of claim 37, wherein the selectable marker is an herbicide resistance gene.

39. The method of claim 37, wherein the bacterial suspension is delivered to the gynoecium of the flower.

40. The method of claim 26, wherein delivering comprises inoculating, spraying, or submerging.

41. The method of claim 26, wherein delivering comprises injecting.

42. The method of claim 26, wherein the bacterial suspension comprises Agrobacterium tumefaciens.

43. The method of claim38, wherein identifying comprises growing the seeds in the presence of an herbicide to which the nucleic acid vector imparts resistance.
